(19) United States
(12) Patent Application Publication
(10) Pub. No.: US 2017/0347731 A1
(43) Pub. Date: Dec. 7, 2017

(54) SYSTEM FOR PROGRESSIVELY CARING FOR EYELASH EXTENSIONS

(71) Applicant: Bella Lash Extensions, LLC, Vineyard, UT (US)

(72) Inventors: Zachary Chipman, Pleasant Grove, UT (US); Haley Chipman, Pleasant Grove, UT (US)

(21) Appl. No.: 15/172,133

(22) Filed: Jun. 2, 2016

(51) Int. Cl. A41G 5/02; A45D 40/26; A61Q 1/10; A61K 8/19; A61K 8/26; A61K 8/58; A61K 8/37; A61K 8/98; A61K 8/97; A61K 8/92; A61K 8/81; A61K 8/73; A61Q 5/12; A61K 8/34

(52) U.S. Cl. CPC A41G 5/02; A61Q 5/12; A45D 40/265; A61Q 1/10; A61K 8/19; A61K 8/26; A61K 8/34; A61K 8/375; A61K 8/985; A61K 8/97; A61K 8/927; A61K 8/92; A61K 8/8176; A61K 8/8158; A61K 8/735; A61K 8/731; A61K 8/73; A61K 8/585; A61K 2800/43; A61K 2800/48; A61K 2800/524; A61K 2800/594; A61K 2800/74

(57) ABSTRACT

A system for maintaining eyelash extensions includes a plurality of components for use throughout the life of a set of eyelash extensions. These components may include a sealer formulated for use just after the extensions have been applied, a sealer and thickener formulated for use after eyelash extensions have started to fall out and a thickener formulated for use after the eyelash extensions have thinned considerably (i.e., few are remaining) and until the individual can have her eyelash extensions filled or replaced. Components of the system that are formulated for initial use and through the middle of the lifetime of the eyelash extensions (e.g., the sealer and the sealer/thickener) may be formulated to nourish the eyelashes and to seal each extension and the natural eyelash to which each extension is adhered. As the lifetime of the eyelash extensions progresses, components of the system may be formulated to provide eyelashes with an increased appearance of volume as more and more of the extensions fall out. Methods for maintaining the appearance of eyelash extensions throughout the lash cycle are also disclosed, and include applying compositions that are tailored for use during various phases of the lash cycle to the eyelash extensions and natural eyelashes.

### TECHNICAL FIELD

This disclosure relates generally to compositions that have been formulated for use with eyelash extensions and, more specifically, to systems that include multiple compositions that have been formulated for use with eyelash extensions and natural eyelashes at different phases, or stages, throughout the lifetime of a set of eyelash extensions, which is also referred to as a “lash cycle.”

### RELATED ART

An eyelash extension typically comprises an individual lash or small group of lashes that are secured to individual natural eyelash of an individual. The eyelash extension is usually secured to the natural eyelash with an adhesive material. Over time, the eyelash extension falls off of its corresponding natural eyelash, or falls out of the individual's eyelashes, as the adhesive material deteriorates or as new growth replaces an older natural eyelash to which the eyelash extension has been secured. A noticeable number of the eyelash extensions that have been applied to an individual's natural lashes will typically begin falling out about one or two weeks after they have been applied. After about three weeks (e.g., three weeks to six weeks), the eyelash extensions have usually thinned appreciably and noticeably. Accordingly, many individuals have their eyelash extensions filled or redone about once every three to six weeks.

Some individuals use mascara to touch up their eyelash extensions. The use of conventional mascaras on eyelash extensions may make the individual's eyelashes appear too full and unnatural. The use of mascaras on eyelashes that already appear to be full because of the eyelash extensions—even when some or many of the eyelash extensions have shed—may make the eyelashes look like “goopy” or “clumpy” mascara has been applied to them, even if the mascara was not goopy or clumpy when applied.

Furthermore, conventional mascaras often include ingredients, such as glycols, oils, and oil-based materials, that damage the adhesive materials that are typically used to secure eyelash extensions to natural eyelashes. Many conventional mascaras are also formulated in a way that makes them difficult to remove from eyelashes. Often, the removal of conventional mascaras requires the use of lotions, creams or soaps, as well as significant mechanical effort, all of which may deteriorate and/or weaken the adhesive material that bonds each eyelash extension to a natural eyelash. For these reasons, the use of conventional mascaras may weaken the adhesive material and cause eyelash extensions to shed prematurely.

### SUMMARY

A system for maintaining eyelash extensions may include a number of components that are configured for use at different phases, or stages, throughout the lifetime, or lash cycle, of a set of eyelash extensions. The components of such a system may be formulated to extend the lifetime of the eyelash extensions and to maintain a somewhat consistent appearance throughout the lifetime of the eyelash extensions. The system may include a series of sealing and/or volumizing mascaras that are formulated to be used in harmony with the lash cycle for a set of eyelash extensions; each component of the system is more voluminous than the previous component to accommodate the replacement of older eyelashes by new growth and the shedding of eyelash extensions. Each component of such a system may be free or substantially free of ingredients (e.g., glycols, oils, oil-based materials, etc.) that weaken the adhesive materials that are used to secure eyelash extensions to natural eyelashes. Components of the system that add color to lashes may also be formulated for removal with a gentle cleanser that will not damage the eyelash extensions or the adhesive material that secures them to natural lashes. In some embodiments, a system according to this disclosure may also include a gentle cleanser.

Each component of such a system may include a bottle and lid for containing the component and a brush (e.g., a mascara brush) for applying the component to the individual's natural lashes and to eyelash extensions that have been secured to her natural lashes. In a specific embodiment, a system for maintaining eyelash extensions includes a sealer, a sealer/thickener, and a thickener. Of course, embodiments of systems that include any two of the components are also within the scope of this disclosure, as are systems that include more than three components (e.g., a number of sealer/thickeners having varying degrees of mascara thickness, various types and/or thicknesses of mascaras, etc.). These components may be packaged together to provide a convenient system for maintaining the look of new eyelash extensions throughout the entire lash cycle.

The sealer may be formulated for use during an initial phase, or stage, of the lifetime of the eyelash extensions, in which 100% to 70% of the eyelash extensions that were originally applied remain in place (e.g., during the first and second weeks after application of the eyelash extensions, during the first week after application of the eyelash extensions, etc.). The sealer may include nutrients to maintain or enhance the health of (i.e., condition) the individual's natural lashes, strengthen the individual's natural lashes, and promote the growth of healthy, strong new eyelashes. In some embodiments, the sealer may be formulated to reinforce the adhesive bond between each eyelash extension and its corresponding natural eyelash. In some embodiments, the sealer may include a colorant (e.g., black, brown, auburn, etc.). The sealer may also form a protective coat over each natural lash and over each eyelash extension, which may increase the longevity of each eyelash extension, as well as the length of time the adhesive material may retain each eyelash extension on a natural eyelash. Such a component may be formulated for daily application (e.g., once daily, etc.). In addition to referring to such a component as a sealer, it may also be referred to as a “minimum sealer” due to the minimal coverage in thickness it provides over each eyelash, and as an “initial component.”

The sealer/thickener may be formulated for use during an intermediate phase, or stage, of the lifetime of the eyelash extensions, in which fewer than 70% down to 40% of the eyelash extensions that were originally applied remain in place (e.g., during the third week after application of the eyelash extensions, during the second and third weeks after application of the eyelash extensions, etc.). The sealer/thickener may be formulated to provide at least some of the same functionality (e.g., provide nutrients, seal, etc.) as the sealer, as well as color and a so-called “volumizing” effect. Thus, the sealer/thickener may mitigate at least some of the loss in eyelash volume that occurs as eyelash extensions start to fall out, or shed. In some embodiments, the sealer/thickener may have the viscosity of a thin (i.e., low viscosity) mascara. Such a component may be formulated for daily application (e.g., once daily, etc.).

In a more specific embodiment, a sealer/thickener according to this disclosure may include nutrients for the natural lashes, at least one conditioner for the natural lashes, a binder for forming a film on the lash extensions, the adhesive material and the natural lashes, and a thickener for adding volume and color to the lash extensions and the natural lashes. As the binder coats the eyelash extensions, the adhesive material and the natural lashes, the binder may hold the nutrients and the at least one conditioner in contact with the natural lashes.

The sealer/thickener may also be referred to as a “medium sealer/thickener” due to the intermediate coverage in thickness it provides over each eyelash, and as an “intermediate component.”

The thickener may be formulated for use during a final phase, or stage, or an end phase, or stage, of the lifetime of the eyelash extensions, in which fewer than 40% of the eyelash extensions remain (e.g., during the fourth week after application of the eyelash extensions, during the fourth and fifth weeks after application of the eyelash extensions, etc.). In the final phase, since many of the eyelash extensions have fallen out or are close to falling out, and they may soon be replaced, the thickener is formulated to provide the individual's natural eyelashes and her remaining extensions with a full, voluminous look, while continuing to preserve the remaining extensions. Accordingly, the thickener may include ingredients that coat the natural eyelashes and the eyelash extensions while imparting them with color to enable the individual to retain the full look provided that was provided by newer eyelash extensions until she is able to receive new eyelash extensions. In some embodiments, the thickener may have the viscosity of a medium or thick (i.e., medium viscosity, high viscosity, etc.) mascara. Such a component may be formulated for daily application (e.g., once daily, etc.). In addition to referring to such a component as a thickener, it may also be referred to as a “maximum thickener” due to the maximal coverage in thickness it provides over each eyelash, and as a “final component” of the system.

Methods for maintaining natural eyelashes and eyelash extensions throughout the lifetime of the eyelash extensions are also disclosed. Such a method includes maintaining the adhesive bonds between the eyelash extensions and the natural eyelashes to which they are secured, sealing the natural eyelashes and eyelash extensions, adding a more voluminous look to the eyelashes after eyelash extensions begin falling out, and increasing the voluminous look of the eyelashes as more of the eyelash extensions fall out.

Other aspects, as well as features and advantages of various aspects, of the disclosed subject matter will become apparent to those of ordinary skill in the art through consideration of the ensuing description, the accompanying drawings and the appended claims.

### BRIEF DESCRIPTION OF THE DRAWINGS

In the drawings:

FIG. 1 illustrates the placement of an eyelash extension on a natural eyelash;

FIG. 2 provides a representation of a system for progressively caring for eyelash extensions throughout the lifetime of the eyelash extensions;

FIG. 3 depicts the elements of each component of the system shown in FIG. 2;

FIGS. 4 and 4A depict the application of a sealer to new eyelash extensions at an initial phase during the lifetime of the eyelash extensions, with FIG. 4 showing the eyelashes before application of the sealer and FIG. 4A showing the eyelashes after the sealer has been applied;

FIGS. 5 and 5A show the application of a sealer/thickener to eyelashes, including natural eyelashes and eyelash extensions, from which eyelash extensions have begun to shed; i.e., during an intermediate phase of the lifetime of the eyelash extensions, with FIG. 5 showing the eyelashes before application of the sealer/thickener and FIG. 5A showing the eyelashes after the sealer/thickener has been applied; and

FIGS. 6 and 6A illustrate the application of a thickener to natural eyelashes and remaining eyelash extensions during an end phase of the lifetime of the eyelash extensions, with FIG. 6 showing the eyelashes before application of the thickener and FIG. 6A showing the eyelashes after the thickener has been applied.

### DETAILED DESCRIPTION

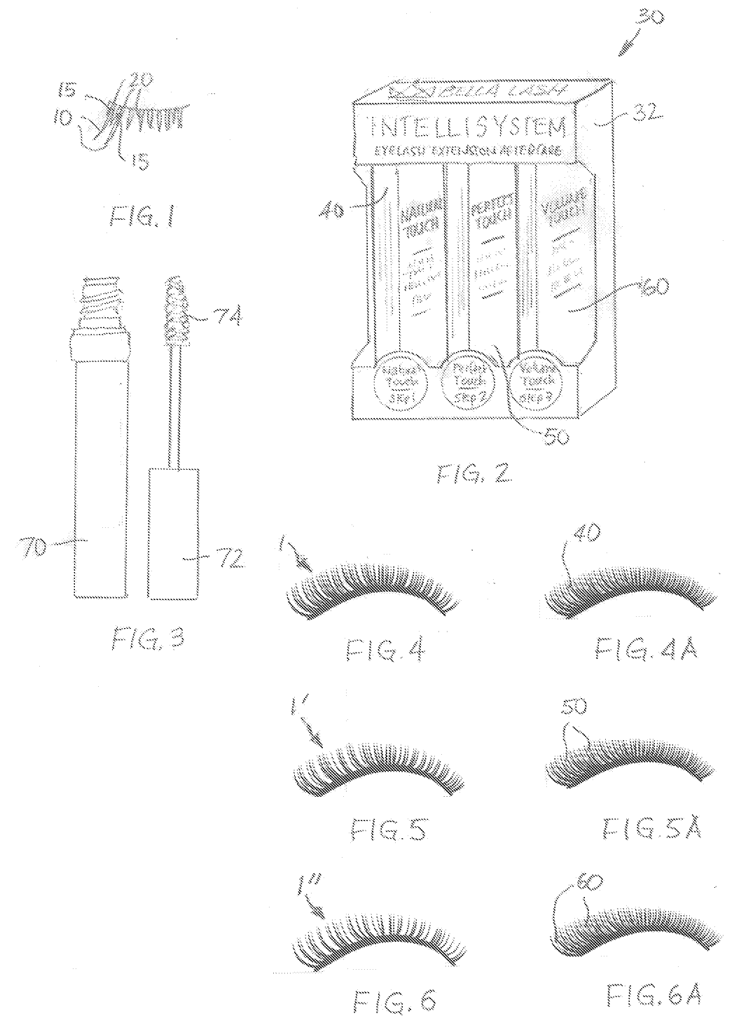

As illustrated by FIG. 1, an eyelash extension 10 may comprise a single lash (as shown) or a small group of lashes (not shown) that may be secured to a natural eyelash 20 on an individual's eyelid with an adhesive material 15. Techniques for applying eyelash extensions 10 to natural eyelashes 20, as well as various adhesive materials 15 for securing the eyelash extensions 10 to natural eyelashes 20 are well known.

Once the eyelash extensions 10 have been secured to natural eyelashes 20, the adhesive material 15 may cure, either on its own or with the aid of a sealer, such as that available from Bella Lash Extensions, LLC, of Vineyard, Utah, as intelliSeal sealer. Such a sealer may be formulated to (i.e., include ingredients in effective amounts that will) encourage or accelerate curing of the adhesive material 15, as well as binders for coating and sealing the eye lashes, including each natural eyelash 20 and the eyelash extension 10 that has been secured thereto. Since the sealer may be washed away or wear off in a relatively short period of time (e.g., a day or two, etc.), the eyelash extensions may still be very new when they are no longer protected.

FIG. 2 illustrates an embodiment of a system 30 for maintaining eyelash extensions 10 (FIG. 1) throughout their expected lifetime. The system 30 includes a package 32 (illustrated as a box) that may hold three primary components: a sealer 40, a sealer/thickener 50, and a thickener 60. As illustrated by FIG. 3, each of these components is stored in a bottle 70 with a lid 72 and an applicator 74 protruding from an interior of the lid 72. The applicator 74 may comprise a brush, such as a curved eyelash brush of the type typically used to apply mascara to an individual's eyelashes. Such a system may also include a gentle cleanser, such as that available from Bella Lash Extensions, LLC, of Vineyard, Utah, as Lash Cleanser, which is an oil-free, water-based cleanser that includes extracts of chamomile and saponaria.

With returned reference to FIG. 1, and with reference to FIG. 2, the sealer 40 of the system 30 is formulated to strengthen natural eyelashes 20 and increase extension life. The sealer 40 includes one or more binders, which form films on and coat eyelash extensions 10. By coating the eyelash extensions 10, the binder may increase their longevity, as well as that of the adhesive material 15, which may increase the length of time the adhesive material 15 may retain each eyelash extension on a natural eyelash 20 and, thus, increase the lifetime of the eyelash extensions 10 and the lash cycle. Since the binder may increase the longevity of the adhesive material 15, the binder may also function as an adhesive support agent. One or more nutrients (e.g., vitamins, plant extracts, etc.) may be included to nourish, condition and/or strengthen the natural lashes 20, as well as promote the growth of healthy, strong new eyelashes. The sealer 40 may also include an adhesive support agent. The adhesive support agent may help fresh adhesive material 15 cure to its maximum potential, may accelerate the rate at which the adhesive material 15 cures and/or may maintain a strength of the adhesive material 15 as the adhesive material 15 and the eyelash extensions 10 age. Optionally, the sealer 40 may include one or more fragrances and/or fragrance absorbing agents to mask and/or absorb fumes from the adhesive material 15 (including uncured adhesive material 15). The sealer 40 may be formulated for compatibility (i.e., safe use) with the eyelash extensions 10; thus, it may lack glycols, oils and oil-based materials and it may be formulated in a manner that enables it to be removed with gentle cleansing (e.g., with a gentle cleanser that will not degrade the adhesive material 15 and without agitating the eyelash extensions 10, the adhesive material 15, or the natural eyelashes).

In a specific embodiment, such a sealer 40 may include water, SDA 40 alcohol, polyvinylpyrrolidone (PVP), Aloe barbadensis (aloe vera) leaf extract, Chondrus crispus (carrageenan/Irish Moss) extract, Equisetum hiemale (Horsetail) extract, xanthan gum, sodium hyaluranate and phenoxyethanol. The water carries the other ingredients of the sealer 40. The alcohol serves as an adhesive support agent.

The PVP is a water-soluble synthetic polymer that functions as a binder, which may form a film on and, thus, coat the eyelash extensions 10, the adhesive material 15, and the natural eyelashes 20. Since the binder forms a film that coats the eyelash extensions 10, the adhesive material 15, and the natural eyelashes 20, the binder seals the eyelash extensions 10, the adhesive material 15, and the natural eyelashes 20.

The aloe vera leaf extract, the Irish Moss extract and the Horsetail extract are natural extracts that include vitamins and other nutrients and, thus, provide nutrients to the natural lashes 20. Sodium hyaluronate, which is the sodium salt of hyaluronic acid, a naturally occurring polysaccharide found in connective tissues, conditions the natural lashes 20 and, thus, acts as a conditioner. The nutrient(s) and/or the conditioner(s) may be dispersed throughout the binder; thus, as the binder forms a coating on an eyelash extension 10, adhesive material 15, and a natural eyelash 20, the nutrient(s) and/or the conditioner(s) may contact, nourish and condition the natural eyelash 20.

The phenoxyethanol serves as a fragrance and/or as a preservative.

Xanthan gum is a thickening agent, which is used to control the viscosity of the sealer 40.

In some embodiments, the sealer 40 may also act as a low viscosity (i.e., thin) mascara. Such an embodiment of sealer 40 may include a colorant; for example, one or more pigments to impart it with a desired color (e.g., black, brown, auburn, etc.). Additionally, a sealer 40 may include one or more thickeners.

A specific embodiment of a sealer 40 that seals and acts as a low viscosity mascara includes water, alcohol, PVP, Equisetum hiemale (Horsetail) extract, bentonite clay, phenoxyethanol, acrylates/octylacrylamide copolymer, caprylic/capric triglycerides, Aloe barbadensis (aloe vera) leaf juice, algae extract (e.g., Chondrus crispus (carrageenan/Irish Moss) extract, etc.), triethanolamine, hydroxypropyl methylcellulose, sodium hyaluranate and, optionally, iron oxides.

The water of the sealer 40 carries the other ingredients of such an embodiment of the sealer 40. The alcohol serves as an adhesive support agent.

The PVP functions as a binder and will form a film that coats the natural lashes 20, the adhesive material 15, and the eyelash extensions 10. The acrylates/octylacrylamide copolymer, which is a copolymer of octylacrylamide and one or more monomers consisting of acrylic acid, methacrylic acid or one of their simple esters, also acts as a binder. Hydroxypropyl methylcellulose is a semisynthetic polymer based on cellulose, and may also function as a binder. Again, since a binder forms a film that coats the eyelash extensions 10, the adhesive material 15, and the natural eyelashes 20, the binder seals the eyelash extensions 10, the adhesive material 15, and the natural eyelashes 20.

The Horsetail extract, the aloe vera leaf juice, and the algae extract are natural extracts that include vitamins and other nutrients and, thus, provide nutrients to the natural lashes 20. Caprylic/capric triglycerides are a mixed triester of glycerin and caprylic and capric acids that may condition, or function as a conditioner for, the natural lashes 20. The sodium hyaluronate may also condition, or function as a conditioner for, the natural lashes 20. When dispersed throughout the binder, the nutrient(s) and/or the conditioner(s) may contact, nourish and condition the natural eyelash 20 as the binder(s) form(s) a coating on an eyelash extension 10, adhesive material 15, and a natural eyelash 20.

The bentonite clay helps the mascara dry quickly and may serve as a thickener. The algae extract and the hydroxypropyl methylcellulose may also increase the viscosity of the sealer 40.

Triethanolamine may serve as a surfactant to emulsify non-soluble particles in the sealer 40. The hydroxypropyl methylcellulose may stabilize the emulsion.

Triethanolamine may also be used to adjust the pH (i.e., acidity/alkalinity) of the sealer 40 due to its strong alkalinity.

The iron oxides may impart the sealer 40 with a desired color.

As illustrated by FIGS. 4 and 4A, the sealer 40 may be applied to eyelashes 1, including eyelash extensions 10 (FIG. 1), adhesive material 15 (FIG. 1), and natural eyelashes 20 (FIG. 1), during the initial phase of the lash cycle (e.g., while at least 70% of the eyelash extensions 10 remain, etc.) (e.g., during the first week of the lash cycle, during the first and second weeks of the lash cycle, etc.). The sealer 40 may be applied to the tops and bottoms of the lashes 1 with an applicator 74 (FIG. 3) once each day or twice a day.

Returning reference again to FIGS. 1 and 2, the sealer/thickener 50 of the embodiment of the system 30 depicted by FIG. 2 may be formulated for use once an individual has started to shed eyelash extensions 10 (i.e., during the intermediate phase of the lash cycle). The sealer/thickener may be formulated to seal the remaining eyelash extensions 10, which may prolong or increase the lifetime of the remaining eyelash extensions 10. In addition, the sealer/thickener 50 may be formulated to enhance the look of the individual's remaining eyelash extensions 10, and of her natural eyelashes 20, or to volumize the remaining eyelash extensions 10 and the natural eyelashes 20. Thus, the sealer/thickener 50 may be thicker than, or more viscous than, the sealer 40. The sealer/thickener 50 may be formulated for compatibility (i.e., safe use) with the eyelash extensions 10; thus, it may lack glycols, oils and oil-based materials and it may be formulated in a manner that enables it to be removed with gentle cleansing (e.g., with a gentle cleanser that will not degrade the adhesive material 15 and without agitating the eyelash extensions 10, the adhesive material 15, or the natural eyelashes).

A specific embodiment of the sealer/thickener 50 includes water, alcohol, PVP, Equisetum hiemale (Horsetail) extract, bentonite clay, phenoxyethanol, acrylates/octylacrylamide copolymer, caprylic/capric triglycerides, Aloe barbadensis (aleo vera) leaf juice, algae extract (e.g., Chondrus crispus (carrageenan/Irish Moss) extract, etc.), triethanolamine, hydroxypropyl methylcellulose, sodium hyaluranate, and, optionally, iron oxides. Notably, these are the same ingredients that are present in an above-described embodiment of the sealer 40, albeit with greater amounts of one or more thickeners (e.g., bentonite clay, hydroxypropyl methylcellulose, etc.) to render the sealer/thickener 50 more like mascara than the sealer 40.

Application of the sealer/thickener 50 to eyelashes 1′ that are in the intermediate phase of the lash cycle (e.g., while at least 40% but fewer than 70% of the eyelash extensions 10 remain, etc.) (e.g., during the second and third weeks of the lash cycle, during the third week of the lash cycle, etc.) is depicted by FIGS. 5 and 5A. The sealer/thickener 50 may be applied to the tops and bottoms of the lashes 1′ with an applicator 74 (FIG. 3) once each day or twice a day.

With reference once again returned to FIGS. 1 and 2, the thickener 60 of the embodiment of system 30 depicted by FIG. 3 may be even thicker (i.e., more viscous) and mascara-like than the sealer/thickener 50. The formulation of the thickener 60 may enable it to blend with the individual's natural eyelashes 20 and remaining eyelash extensions 10 to provide a good boost of color to fill in areas that have been left sparse from the loss, or shedding, of eyelash extensions 10. The thickener 60 may also be formulated for compatibility (i.e., safe use) with the eyelash extensions 10 and for easy removal; thus, it may lack glycols, oils and oil-based materials and it may be formulated in a manner that enables it to be removed with gentle cleansing (e.g., with a gentle cleanser that will not degrade the adhesive material 15 and without agitating the eyelash extensions 10, the adhesive material 15, or the natural eyelashes).

In a specific embodiment, the thickener 60 may include water, beeswax, stearic acid, ozokerite wax, carnauba wax, Equisetum hiemale (Horsetail) extract, alcohol, cyclomethicone, PVP, acrylates/octylacrylamide copolymer, Aloe barbadensis (aloe vera) leaf juice, algae extract (e.g., Chondrus crispus (carrageenan/Irish Moss) extract, etc.), phenoxyethanol, magnesium aluminum silicate, triethanolamine, and, optionally, iron oxides.

The water of the thickener 60 carries the other ingredients of such an embodiment of the thickener 60. The alcohol and the cyclomethicone (a silicon-based compound) may also serve as solvents.

The beeswax (purified wax from honeycomb), the ozokerite wax (a hydrocarbon wax derived from mineral sources or from petroleum), and/or the carnauba wax may increase the viscosity of the thickener 60 and, thus, contribute to the thickness of the thickener 60.

The PVP, the beeswax, the ozokerite wax, and/or the acrylates/octylacrylamide may function as binders and will form films that coat the natural lashes 20, the adhesive material 15, and the eyelash extensions 10. To repeat: because the binder forms a film that coats the eyelash extensions 10, the adhesive material 15, and the natural eyelashes 20, the binder seals the eyelash extensions 10, the adhesive material 15, and the natural eyelashes 20.

Horsetail extract, the aloe vera leaf juice, and the algae extract are natural extracts that include vitamins and other nutrients and, thus, provide nutrients to the natural lashes 20. The cyclomethicone is an emollient and may, therefore, condition the natural lashes 20, or function as a conditioner. The horsetail extract, the aloe vera leaf juice, the algae extract, and/or the cyclomethicone, when dispersed throughout the binder, may contact, nourish and condition the natural eyelash 20 as the binder(s) form(s) a coating on an eyelash extension 10, adhesive material 15, and a natural eyelash 20.

The beeswax, the stearic acid, the ozokerite wax, the algae extract and/or the cyclomethicone may impart the thickener 60 with its thickness, or viscosity. Notably, the thickener 60 includes more viscosity-increasing components than the sealer/thickener 50, imparting the thickener 60 with a greater ability than the sealer/thickener 50 to volumize lashes that are in the final phase of the lash cycle.

The beeswax, the stearic acid, the ozokerite wax, the algae extract and/or the cyclomethicone may also serve as surfactants and/or stabilize the emulsion that defines the thickener 60. Triethanolamine may serve as a surfactant to emulsify non-soluble particles in the thickener 60.

Since Triethanolamine is a strongly alkaline substance, it may also be used to adjust the pH (i.e., acidity/alkalinity) of the thickener 60.

The beeswax, the stearic acid, and/or the phenoxyethanol may act as fragrances that contribute to the aroma of the thickener 60.

The phenoxyethanol may also function as a preservative.

The iron oxides may impart the thickener 60 with a desired color.

With reference to FIGS. 6 and 6A, the thickener 60 may be applied to eyelashes 1″ that are in the end phase, or final phase, of the lash cycle (e.g., when fewer than 40% of the eyelash extensions 10 remain, etc.) (e.g., during and/or after the fourth week of the lash cycle, etc.). An applicator 74 (FIG. 3) may be used to apply the thickener 60 to the tops and bottoms of the lashes 1″ once each day or as needed or desired.

Since the sealer/thickener 50 and the thickener 60 may be formulated to be removed with a gentle cleanser, both of these components may be periodically removed from an individual's lashes without causing the removal of any additional eyelash extensions 10 (FIG. 1) from the individual's natural eyelashes 20 (FIG. 1). Without limitation, a gentle cleanser may be applied onto a suitable substrate (e.g., a cotton round, a soft cloth, etc.) and then run gently across the lashes.

Although the preceding disclosure provides many specifics, these should not be construed as limiting the scope of any of the ensuing claims. Other embodiments may be devised which do not depart from the scopes of the claims. Features from different embodiments may be employed in combination. The scope of each claim is, therefore, indicated and limited only by its plain language and the full scope of available legal equivalents to its elements.

What is claimed:

1. A system for maintaining eyelash extensions, comprising:
a minimum sealer formulated for use with new lash extensions that have been adhered to natural lashes to maintain the new lash extensions during an initial phase of a lash cycle;
a medium sealer/thickener formulated for use with natural lashes and lash extensions during an intermediate phase of the lash cycle, once the lash extensions have started shedding to maintain remaining lash extensions, to coat the remaining lash extensions and the natural lashes, and to make the natural lashes and the lash extensions appear thicker and/or more voluminous; and
a maximum thickener formulated for use with natural lashes and lash extensions during a final phase of the lash cycle, after a majority of the lash extensions have shed to coat the remaining lash extensions and the natural lashes and to make the natural lashes and the remaining lash extensions appear thicker and more voluminous,
the medium sealer/thickener and the maximum thickener being free of ingredients that weaken adhesive material securing the lash extensions to the natural lashes and being formulated for removal with a gentle cleanser.

2. The system of claim 1, wherein the minimum sealer is formulated for use while at least 70% of the eyelash extensions remain.

3. The system of claim 2, wherein the medium sealer/thickener is formulated for use while at least 40% but fewer than 70% of the eyelash extensions remain.

4. The system of claim 3, wherein the maximum thickener is formulated for use when fewer than 40% of the eyelash extensions remain.

5. The system of claim 1, wherein the minimum sealer includes:
nutrients for the natural lashes;
at least one conditioner for the natural lashes;
an adhesive support agent for further curing and/or maintaining the adhesive material securing the lash extensions to the natural lashes; and
a binder for coating a film on the lash extensions, the adhesive material and the natural lashes.

6. The system of claim 5, wherein the minimum sealer further includes:
a colorant.

7. The system of claim 6, wherein the colorant comprises at least one iron oxide.

8. The system of claim 5, wherein the nutrients comprise Equisetum hiemale extract, aloe vera, and/or Irish Moss.

9. The system of claim 5, wherein the adhesive support agent comprises alcohol.

10. The system of claim 5, wherein the binder comprises polyvinylpyrrolidone, acrylates/octylacrylamide copolymer, and/or hydroxypropyl methylcellulose.

11. The system of claim 1, wherein the medium sealer/thickener includes:
nutrients for the natural lashes;
an adhesive support agent for maintaining adhesive securing the lash extensions to the natural lashes;
a binder; and
a thickener.

12. The system of claim 11, wherein the thickener comprises Irish Moss, carnauba wax and/or bentonite clay.

13. The system of claim 1, wherein the maximum thickener includes:
nutrients for the natural lashes;
a binder; and
a thickener.

14. The system of claim 13, wherein the thickener comprises stearic acid, ozokerite, cyclomethicone, Irish Moss, carnauba wax and/or bentonite clay.

15. The system of claim 1, wherein the maximum thickener further includes magnesium aluminum silicate.

16. A method for maintaining eyelash extensions, comprising:
applying a sealer periodically to the eyelash extensions, natural eyelashes to which the eyelashes are secured, and an adhesive material that secures the eyelash extensions to the natural eyelashes during an initial phase of a lash cycle, while the lash extensions are new;
applying a sealer/thickener periodically to remaining eyelash extensions, the natural eyelashes, and remaining adhesive material during an intermediate phase of the lash cycle, in which lash extensions have started to fall out of an individual's lashes;
periodically removing the sealer/thickener without causing additional lash extensions to fall out of the individual's lashes;
applying a thickener periodically to remaining eyelash extensions, remaining adhesive material, the natural eyelashes, and any new natural eyelashes during a final phase of the lash cycle, in which a loss of eyelash extensions has resulted in sparse areas in the individual's lashes; and
periodically removing the thickener without causing additional lash extensions to fall out of the individual's lashes.

17. The method of claim 16, wherein applying the sealer periodically, applying the sealer/thickener periodically and/or applying the thickener periodically comprises applying the sealer, the sealer/thickener and/or the thickener at least once each day during the respective initial phase, intermediate phase and/or final phase of the lash cycle.

18. The method of claim 16, wherein applying the sealer includes applying the sealer periodically while at least 70% of the lash extensions remain.

19. The method of claim 16, wherein applying the sealer/thickener includes applying the sealer/thickener while at least 40%, but fewer than 70% of the lash extensions remain.

20. The method of claim 16, wherein applying the thickener includes applying the thickener when fewer than 40% of the lash extensions remain.

21. The method of claim 22, wherein applying the thickener optionally includes applying the thickener after the fourth week of the lash cycle, until an end of the lash cycle.

22. A sealer/thickener formulated for use with natural lashes and lash extensions once the lash extensions have started shedding to maintain the remaining lash extensions, to coat the remaining lash extensions and the natural lashes, and to make the natural lashes and the lash extensions appear thicker and/or more voluminous, the sealer/thickener comprising:
nutrients for the natural lashes;
at least one conditioner for the natural lashes;
a binder for forming a film on the lash extensions, the adhesive material and the natural lashes, the binder formulated to hold the nutrients and the at least one conditioner in contact with the natural lashes; and
a thickener for adding volume and color to the lash extensions and the natural lashes.
